(19) United States
(12) Patent Application Publication
(10) Pub. No.: US 2025/0090985 A1
(43) Pub. Date: Mar. 20, 2025

(54) ULTRASONICALLY BONDED ELECTRET FILTER MEDIA

(71) Applicant: DelStar Technologies, Inc., Alpharetta, GA (US)

(72) Inventors: Yasar Kiyak, Alpharetta, GA (US); Andrew G. Platt, Middletown, DE (US); Andrew Scarff, Alpharetta, GA (US)

(21) Appl. No.: 18/826,878

(22) Filed: Sep. 6, 2024

Related U.S. Application Data
(60) Provisional application No. 63/582,888, filed on Sep. 15, 2023.

(51) Int. Cl. B01D 39/16; A61M 16/06; A61M 16/10; A62B 23/02; B01D 46/00; B01D 46/52

(52) U.S. Cl. CPC B01D 39/163; A61M 16/06; A61M 16/105; B01D 46/0032; B01D 46/521; A61M 2205/7545; A62B 23/025; B01D 2239/0216; B01D 2239/0435; B01D 2239/0618; B01D 2239/0627; B01D 2239/0636; B01D 2239/064; B01D 2239/0654; B01D 2239/0672; B01D 2239/10; B01D 2239/1258; B01D 2239/1291; B01D 2275/10; B01D 2279/40

(57) ABSTRACT

Filter media and filters are provided having a fibrous web including first and second dissimilar electret fibers ultrasonically bonded to each other. The first and second electret fibers may be directly bonded to each other without needle punching, which substantially maintains the thickness or loft of the fibrous web of the filter media and filters. Maintaining the thickness or loft of the fibrous web during the bonding process decreases the pressure drop across the filter media and increases its dust holding capacity. Medical grade filters and/or self-supporting (i.e., self-pleatable) air filters are also provided. The medical grade filters and/or self-supporting air filters may include one or more support layers ultrasonically bonded to the fibrous web or fiber layers without needle punching, which maintains the stiffness of the support layers and allows the filter to be produced in a single manufacturing step.

### CROSS-REFERENCE TO RELATED APPLICATIONS

This application claims priority to U.S. Provisional Patent Application No. 63/582,888 filed on Sep. 15, 2023, the contents of which are incorporated herein by reference to the extent consistent with the present disclosure.

### TECHNICAL FIELD

This description generally relates to filtration media and filters, and more particularly to filter media that includes at least two dissimilar electret fibers ultrasonically bonded together.

### BACKGROUND

Liquid and gas filters trap contaminants of many different types from the air, water, or others. Air filters, for example, typically include a filtration media comprising fibrous or porous materials that remove solid particulates, such as dust, pollen, mold, and bacteria from the air.

Depth filters are commonly employed in air filtration devices with a moderate to high efficiency, a low-pressure drop, and a relatively high dust loading capacity. Depth filters generally employ various kinds of fibers that may be formed into a web or other nonwoven structure having tortuous paths between the fibers through which a gas stream, such as air, is passed. The particulate matter in the gas flowing through the paths in the web is retained on the upstream side of the web, or within the tortuous paths of the web due to the size of the particles relative to the paths' diameters.

It is also known to charge various blends of fibers electrostatically to further retain particulate matter through electrostatic attractions between the fibers and the particles. Electrostatic fibers are commonly used in many filtration applications such as face masks and high-efficiency filters to filter submicron contaminants, such as viruses and others. Electrostatically charged filter media are described in U.S. Pat. Nos. 10,571,137 and 9,802,187, the complete disclosures of which are incorporated herein by reference in their entirety for all purposes.

The electrostatic charge may be applied in a variety of ways, such as oxidatively treating the fibers, passing the fibers through a corona (“corona discharge”), hydrocharging, electrostatic fiber spinning, triboelectric charging, or other known methods. Triboelectric charging is a type of contact electrification in which certain materials become electrically charged after they come into contact with a dissimilar material (such as through rubbing) and are then separated.

Electrostatic or “electret” filter media have increased efficiency without necessarily increasing the amount of force required to push the air through the filter media. The “pressure drop” of media is the decrease in pressure from the upstream side of the media to the downstream side. The more difficult it is to force air through the media, the greater the pressure drop, and the greater use of energy to force air through the media. Therefore, it is generally advantageous to decrease pressure drop or maintain pressure drop while increasing the filter's ability to capture contaminants.

Electrostatic filter media is typically created by carding and needle punching two dissimilar electret fiber layers, such as polypropylene (PP) and acrylic, into a fully homogenous material. This manufacturing process provides a three-dimensional web structure with relatively high porosity and low filtration resistance to airflow. In certain cases, a supported filter media, such as a pleated air filter or a medical grade filter, may be created by needle punching a rigid support layer, termed a “scrim”, and then bonding the scrim to the fiber web.

### BRIEF SUMMARY

The following presents a simplified summary of the claimed subject matter in order to provide a basic understanding of some aspects of the claimed subject matter. This summary is not an extensive overview of the claimed subject matter. It is intended to neither identify critical elements of the claimed subject matter nor delineate the scope of the claimed subject matter. Its sole purpose is to present some concepts of the claimed subject matter in a simplified form as a prelude to the more detailed description that is presented later.

Filter media and filters are provided that may include first and second dissimilar electret fibers ultrasonically bonded to each other to form a fibrous web. The fibers may be directly bonded to each other without needle punching, which substantially maintains the thickness or loft of the fiber web in the final filter product. Maintaining the thickness or loft of the fiber web during the bonding process decreases the pressure drop across the filter media and increases its dust-holding capacity. Medical grade filters and/or self-supporting (i.e., self-pleatable) air filters are also provided that may include one or more support layers ultrasonically bonded to the fiber web without needle punching, which maintains the stiffness of the support layers and allows the filter to be produced in a single manufacturing step.

In one aspect, a filter media may include first and second dissimilar electret fibers capable of or configured for triboelectric charge. The first and second fibers may be ultrasonically bonded together at an attachment point to form a fibrous web.

In embodiments, the fibrous web may include a plurality of attachment points. The first and second fibers may be at least partially fused or bonded together at the plurality of attachment points. The attachment points may form indentations in an outer surface of the web.

The first and second fibers may be non-needle punched fibers, i.e., fibers that have not been subjected to the process of mechanical interlocking with a needle, barb or similar device. This substantially maintains the thickness or loft of the fiber web in the resulting filter media. The fiber web in the filter product may have a thickness of at least about 50% greater than a similar conventional fiber web that has been needle punched, or at least about 70%, or at least about 90% of the thickness of a conventional fiber web.

In embodiments, the filter media may have a loftiness of about 50 to about 400 mils. This “high loft” material means that the volume of void space may be greater than the volume of the total solid.

In embodiments, the filter media may have a pressure drop of about 0.1 mmH₂O to about 4 mmH₂O at 10.5 fpm face velocity and a predicted MERV rating of at least about 11 to about 16.

The fibers may be artificial or natural. Suitable materials for the fibers include, but are not limited to, polyesters, such as, polyethylene terephthalate (PET), polyethylene naphthalate (PEN), polycyclohexylenedimethylene terephthalate (PCT), polybutylene terephthalate, or the like, polypropylene (PP), co-polyamides, polyethylene, high density polyethylene (“HDPE”), linear low-density polyethylene (LLDPE), cross-linked polyethylene, polycarbonates, polyacrylates, polyacrylonitriles, acrylic fiber, polylactic acid, polyfumaronitrile, polystyrenes, styrene maleic anhydride, polymethylpentene, cyclo-olefinic copolymer or fluorinated polymers, polytetrafluoroethylene, perfluorinated ethylene and hexfluoropropylene or a copolymer with polyvinylidene fluoride (PVDF), such as P(VDF-TrFE) or terpolymers like P(VDF-TrFE-CFE), propylene, polyimides, polyether ketones, cellulose ester, nylon and polyamides, polymethacrylic, poly(methyl methacrylate), polyoxymethylene, polysulfonates, acrylic, modacrylic, styrenated acrylics, pre-oxidized acrylic, fluorinated acrylic, vinyl acetate, vinyl acrylic, ethylene vinyl acetate, styrene-butadiene, ethylene/vinyl chloride, vinyl acetate copolymer, latex, polyester copolymer, carboxylated styrene acrylic or vinyl acetate, epoxy, acrylic multipolymer, phenolic, polyurethane, cellulose, styrene, or the like, or any combination thereof. Other conventional fiber materials are contemplated.

The fibers may include biocomponent fibers that include two or more different fibers bonded to each other. The biocomponent fibers may include the same material or different materials. These may be typically formed by extruding two polymers from the same spinneret with both polymers contained within the same filament. Suitable materials for bicomponent fibers include, but are not limited to, polypropylene (PP)/polyethylene (PE), polyethylene terephthalate (PET)/polypropylene (PP), and the like.

In one embodiment, the first fibers may include polypropylene (PP) and the second fibers may include acrylic. In embodiments, the PP fibers may be present in an amount of from about 40% to about 60% by weight of the filter media, preferably about 50%. In one embodiment, the acrylic fibers may be present in an amount of from about 40% to about 60% by weight of the filter media, preferably about 50%.

In some embodiments, the filter media may include one or more support layers ultrasonically bonded to the fiber web. The support layer(s) may be ultrasonically bonded to the fibers without needle punching, which increases the overall stiffness of the filter media. This may allow, for example, a self-supporting or self-pleatable filter to be created without a metal wire mesh or other structure designed to increase the stiffness of the filter.

The support layer may include a substrate, sheet, layer, film, an apertured film, mesh, or other media. The support layer may include a nonwoven substrate that includes a structure of individual fibers or threads that may be interlaid, interlocked, or bonded together. Examples of suitable nonwoven materials include, but are not limited to, fibers, layers, or webs that may be meltblown, spunbond or spunlace, heat-bonded, bonded carded, air-laid, wet-laid, co-formed, stitched, hydraulically entangled or the like. Alternatively, the support layer may include a knitted and/or woven material. The knitted material may include any knitting pattern suitable for the desired application. Suitable knitted materials for filter applications include weft-knit, warp knit, knitted mesh panels, compressed knitted mesh, and the like.

In one embodiment, the support layer may include a woven material, such as a scrim. The scrim layer may have a stiffness suitable for a pleated filter and/or a medical grade filter. In certain embodiments, the scrim layer may have a basis weight of at least about 15 gsm, or at least about 40 gsm, or at least about 50 gsm, or from about 50 gsm to about 110 gsm.

In certain embodiments, the filter media may include a single support layer bonded to an outer surface of the fiber web. In other embodiments, the filter media may include first and second support layers. The first and second support layers may include any of the above materials and may include the same material, or different materials. In certain embodiments, the first and second support layers may each include a scrim layer.

In one embodiment, the first and second support layers may be ultrasonically bonded to opposite surfaces of the fiber web. In another embodiment, the first support layer may be bonded to the fiber web, and the second support layer may be bonded to the first support layer.

In another aspect, a filter media for use in a filter is provided. The filter media may include first and second dissimilar electret fibers that may be ultrasonically bonded to each other to form a web. The filter may be or include but is not limited to, an air filter, self-supporting pleated filter, vacuum bag, cabin air filter, HVAC furnace filter, air intake filter of a gas turbine and/or compressor, panel filter, or a medical grade filter, such as a face mask, CPAP filter, and the like.

In another aspect, a self-supporting filter is provided that may include first and second dissimilar electret fibers ultrasonically bonded with each other to form a fiber web, and a second stiffer layer, such as a scrim, ultrasonically bonded to the fiber web. The scrim may substantially maintain its stiffness during ultrasonic bonding, primarily because there may be no needle punching step. The self-supporting filter may be “self-pleatable”, which means that the filter does not include a metal wire mesh or other supporting structure to form and hold the pleats.

In another aspect, a medical grade filter may be provided, such as a filter for use in a facemask, a continuous positive airway pressure (CPAP) machine or the like. The medical grade filter may include first and second dissimilar electret fibers ultrasonically bonded to each other to form a fiber web. The filter may further include a first layer, such as a scrim, ultrasonically bonded to a first surface of the fiber web and a second layer, such as a scrim, bonded to the second surface of the fiber web. The first and/or the second scrim layer may be bonded to the fiber web at substantially the same time the electret fibers are bonded to each other (i.e., in a single step) to increase the speed and reduce the cost of the overall manufacturing process.

In another aspect, a method of manufacturing a filter media is provided. The method may include providing first electret fibers, providing second, dissimilar electret fibers, and ultrasonically bonding the first and second fibers together to form a fibrous web. The fibers may be directly bonded to, or consolidated with, each other without needle punching the fibers, thereby substantially maintaining the thickness and loft of the fiber web in the final filter media product.

In some embodiments, the method may further include ultrasonically bonding one or more support layers to the fiber web. The support layer(s) may be bonded in the same step as the fibers.

In embodiments, the fibers may be ultrasonically bonded to each other such that the web in the filter product may have a thickness of at least about 50% greater than a similar convention fiber web that has been needle punched, or at least about 70%, or at least about 90% of the thickness of the conventional fiber web.

The recitation herein of desirable objects which are met by various embodiments of the present description is not meant to imply or suggest that any or all of these objects are present as essential features, either individually or collectively, in the most general embodiment of the present description or any of its more specific embodiments.

### BRIEF DESCRIPTION OF THE DRAWINGS

FIG. 1 is a side cross-sectional view of a single web of dissimilar fibers ultrasonically bonded with each other to form a filter media;

FIG. 2 is a side cross-sectional view of an ultrasonically bonded filter media having a fiber web and a first support layer;

FIG. 3 is a side cross-sectional view of an ultrasonically bonded filter media having first and second support layers of a material bonded on either side of a carded fiber web;

FIG. 4 is a side cross-sectional view of an ultrasonically bonded filter media having first and second layers of different materials bonded on either side of a carded fiber web;

FIG. 5 is a side cross-sectional view of an ultrasonically bonded filter media having first and second layers of different materials bonded to one side of a carded fiber web; and

FIG. 6 illustrates a method of ultrasonically bonding dissimilar materials to each other.

### DETAILED DESCRIPTION

This description and the accompanying drawings illustrate exemplary embodiments and should not be taken as limiting, with the claims defining the scope of the present description, including equivalents. Various mechanical, compositional, structural, and operational changes may be made without departing from the scope of this description and the claims, including equivalents. In some instances, well-known structures and techniques have not been shown or described in detail so as not to obscure the description. Like numbers in two or more figures represent the same or similar elements. Furthermore, elements and their associated aspects that are described in detail with reference to one embodiment may, whenever practical, be included in other embodiments in which they are not specifically shown or described. For example, if an element is described in detail with reference to one embodiment and is not described with reference to a second embodiment, the element may nevertheless be claimed as included in the second embodiment. Moreover, the depictions herein are for illustrative purposes only and do not necessarily reflect the actual shape, size, or dimensions of the system or illustrated components.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

Except as otherwise noted, any quantitative values are approximate whether the word “about” or “approximately” or the like are stated or not. The materials, methods, and examples described herein are illustrative only and not intended to be limiting.

As used throughout this disclosure, ranges are used as shorthand for describing each and every value that is within the range. It should be appreciated and understood that the description in a range format is merely for convenience and brevity and should not be construed as an inflexible limitation on the scope of any embodiments or implementations disclosed herein. Accordingly, the disclosed range should be construed to have specifically disclosed all the possible subranges as well as individual numerical values within that range. As such, any value within the range may be selected as the terminus of the range. For example, description of a range such as from 1 to 5 should be considered to have specifically disclosed subranges such as from 1.5 to 3, from 1 to 4.5, from 2 to 5, from 3.1 to 5, etc., as well as individual numbers within that range, for example, 1, 2, 3, 3.2, 4, 5, etc. This applies regardless of the breadth of the range.

Additionally, all numerical values are “about” or “approximately” the indicated value, and take into account experimental error and variations that would be expected by a person having ordinary skill in the art. It should be appreciated that all numerical values and ranges disclosed herein are approximate values and ranges, whether “about” is used in conjunction therewith. It should also be appreciated that the term “about,” as used herein, in conjunction with a numeral refers to a value that may be ±0.01% (inclusive), ±0.1% (inclusive), ±0.5% (inclusive), ±1% (inclusive) of that numeral, ±2% (inclusive) of that numeral, ±3% (inclusive) of that numeral, ±5% (inclusive) of that numeral, ±10% (inclusive) of that numeral, or ±15% (inclusive) of that numeral. It should further be appreciated that when a numerical range is disclosed herein, any numerical value falling within the range is also specifically disclosed.

All references cited herein are hereby incorporated by reference in their entireties. In the event of a conflict in a definition in the present disclosure and that of a cited reference, the present disclosure controls.

Filter media and filters are provided that capture particles. The filters may include, but are not limited to, air filters, self-supporting pleated filters, vacuum bags, cabin air filters, HVAC filters, gas turbine and compressor air intake filters, panel filters, and medical grade filters, such as face masks, CPAP filters and the like. Systems and methods of manufacturing such filters are also provided.

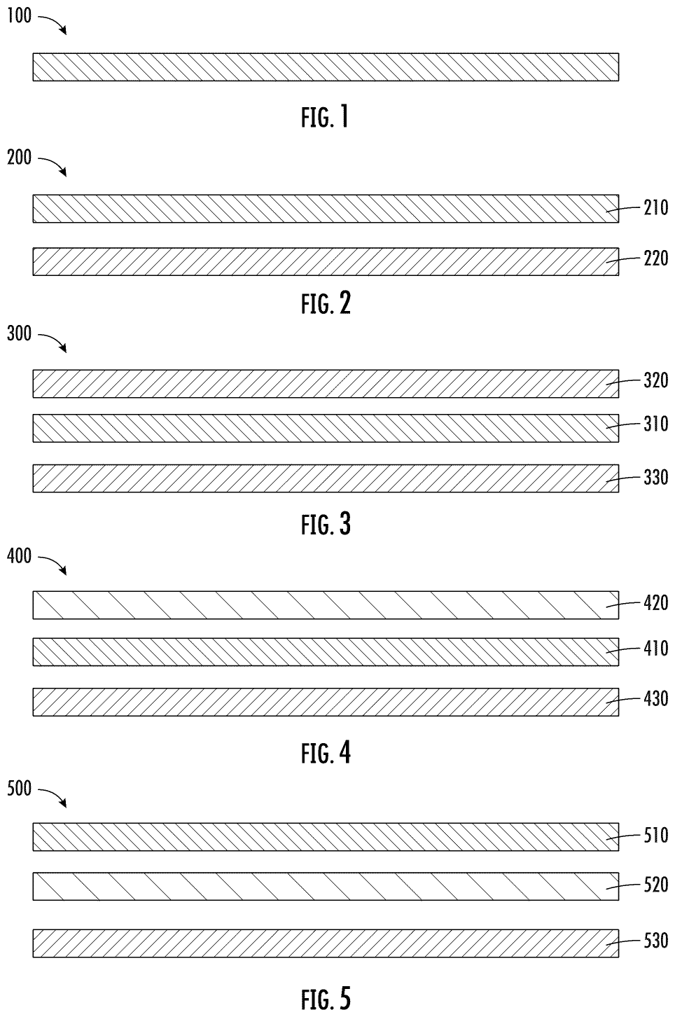

Referring now to FIG. 1, a filter media may include first and second dissimilar fibers that may be ultrasonically bonded to each other to form a single web of fibers 100 that forms the filter media. In certain embodiments, the first and second fibers may be electret fibers. In one such embodiment, the fibers may be triboelectrically charged with each other. The triboelectric effect (also known as triboelectric charging) is a type of contact electrification in which certain materials become electrically charged after they are separated from a different material with which they were in contact. For example, rubbing the two materials with each other increases the contact between their surfaces. The polarity and strength of the charges differ according to the materials, surface roughness, temperature, strain, and other properties.

In certain embodiments, the first fibers may include a tribopositive material and the second fibers may include a tribonegative material or a relatively tribopositive material with a relatively low charge density compared to the first fibers. This increased charge enhances or creates localized electrical field gradients within the filter media to enhance particle removal, thereby enhancing the effectiveness of the filter media.

The fibers may be artificial or natural. Suitable materials for the fibers include, but are not limited to, polypropylene, acrylic, polylactic acid, polyesters, such as polyethylene terephthalate (PET), polyethylene naphthalate (PEN), polycyclohexylenedimethylene terephthalate (PCT), polybutylene terephthalate (PBT), or the like, polypropylene (PP), co-polyamides, polyamides, polyethylene, high density polyethylene (“HDPE”), linear low-density polyethylene (LLDPE), cross-linked polyethylene, polycarbonates, polyacrylates, polyacrylonitriles, polyfumaronitrile, polystyrenes, styrene maleic anhydride, polymethylpentene, cyclo-olefinic copolymer or fluorinated polymers, polytetrafluoroethylene, perfluorinated ethylene and hexfluoropropylene or a copolymer with polyvinylidene fluoride (PVDF), such as P(VDF-TrFE) or terpolymers like P(VDF-TrFE-CFE), propylene, polyimides, polyether ketones, cellulose ester, nylon and polyamides, polymethacrylic, poly(methyl methacrylate), polyoxymethylene, polysulfonates, acrylic, modacrylic, styrenated acrylics, pre-oxidized acrylic, fluorinated acrylic, vinyl acetate, vinyl acrylic, ethylene vinyl acetate, styrene-butadiene, ethylene/vinyl chloride, vinyl acetate copolymer, latex, polyester copolymer, carboxylated styrene acrylic or vinyl acetate, epoxy, acrylic multipolymer, phenolic, polyurethane, cellulose, styrene, or the like, or any combination thereof. Other conventional fiber materials are contemplated.

In embodiments, the first fibers may include polypropylene (PP) and the second fibers may include acrylic fibers. In embodiments, the PP fibers may be present in an amount of from about 40% to about 60% by weight of the filter media, preferably about 50%. In embodiments, the acrylic fibers may be present in an amount of from about 40% to about 60% by the weight of the filter media, preferably about 50%.

In some embodiments, additional fiber types may be introduced into the blend. These fibers may include any of the materials described above, or other materials. In one example, the filter may further include polylactic acid (PLA) fibers, such as Racemic PLLA (poly L-lactide), regular PLLA, poly D-lactide (PLDA), poly-DL-lactic acid (PDLLA), or a combination thereof. In some embodiments, the additional fibers may include a blend of PLA and other materials, such as polyhydroxyalkanoate (PHBV) fibers or the like. Such PLA blends are described in commonly assigned Provisional patent application Ser. No. 63/410,729, filed Sep. 28, 2022, the entire disclosures of which are hereby incorporated by reference herein for all purposes.

The filtration media may include a charge additive to modify the triboelectric charge of the fibers and increase the stability and/or duration of the triboelectric charge in the filter. This increases the overall filtration efficiency of the filter without compromising other important characteristics of the filters, such as longevity, dust holding capacity, and the pressure drop or air flow through the filter. Suitable charge additives for triboelectric charging are described in commonly assigned Provisional patent application Ser. No. 63/410,731, filed Sep. 28, 2022, the entire disclosures of which are hereby incorporated by reference herein for all purposes.

In certain embodiments, the fibers may include a silicone-based coating to improve the efficiency of the filter media at capturing contaminants. The silicone-based coating may include a reactive silicone macroemulsion. The silicone emulsion may include, for example, dimethyl silicone emulsions, amino type silicone emulsions, organo-functional silicone emulsions, resin type silicone emulsions, film-forming silicone emulsions, or the like. In one embodiment, the reactive silicone macroemulsion may include an amino functional polydimethylsiloxane and/or a polyethylene glycol monotridecyl ether. Suitable silicone coatings are described in commonly assigned U.S. Provisional patent application Ser. No. 63/406,686, filed Sep. 14, 2022, the complete disclosure of which is incorporated herein by reference.

The first and second fibers may have thicknesses (diameter) that may be suitable for the application. In some embodiments, the fibers have at least one dimension in the range of from about 1 to about 10,000 micrometers or from about 1 to about 1,000 micrometers or from about 10 to 100 micrometers. In certain embodiments, the fibers may have a diameter of from about 0.1 microns to about 200 microns, or preferably from about 5 microns to about 50 microns.

The thickness of the fibers may also be measured in Denier, which is a unit of measure in the linear mass density of fibers. In some embodiments, the fibers may have a linear density of about 0.5 Denier (D) to about 50 Denier, or about 1 Denier to about 10 Denier. The filter media may include fibers with the same or different linear densities.

The fibers may be either continuous or non-continuous (i.e., staple fibers). In certain embodiments, the fibers may include staple fibers having a length of from about 1 mm to about 200 mm, or from about 5 mm to about 150 mm, and more preferably from about 30 mm to about 80 mm.

In embodiments, the fibers may have a spin finish of about 2% or lower, or about 1% or lower, or no spin finish (e.g., a naked fiber). A spin finish as defined herein may refer to a liquid or solid composition that may be applied to the surfaces of man-made fibers in order to improve the processing of such fibers in short-staple or long-staple spinning.

The fibers contemplated may have any one or more cross-sectional shapes, including without limitation, circular, kidney bean, dog bone, trilobal, barbell, bowtie, star, Y-shaped, or the like, or any combination thereof. These shapes and/or other conventional shapes may be used with the embodiments to obtain the desired performance characteristics. The fibers in the substrate may stay connected to each other through thermal bonds, and chemical bonds, by being entangled with one another, through the use of binding agents, such as adhesives, or the like.

The fibers may include biocomponent fibers that include two or more different fibers bonded to each other. The fibers may include the same material or different materials. These may be typically formed by extruding two polymers from the same spinneret with both polymers contained within the same filament. Suitable materials for bicomponent fibers include, but are not limited to, polypropylene (PP)/polyethylene (PE), polyethylene terephthalate (PET)/polypropylene (PP), and the like.

The fibers may include thermally splittable fibers configured to reduce the fiber size of at least some components of the fibers within the filter media. This reduced fiber size increases the overall efficiency of such filters at capturing contaminants, particularly those contaminants having a size range of from about 0.1 to about 10 microns, without compromising other important characteristics of the filters. In one such embodiment, the fibers may comprise one or more bicomponent fibers each having first and second components. The first component may include a thermoplastic elastomer material and a thermoplastic material and may have a higher shrinkage ratio/percentage/rate than the second component such that at least a portion of the first component separates from the second component upon the application of heat or thermal energy to the fiber. A more complete description of thermally splittable fibers may be found in commonly assigned, co-pending U.S. Provisional Patent Application No. 63/440,517, filed Jan. 23, 2023, the complete disclosure of which is incorporated herein by reference for all purposes.

In some embodiments, the filter media may include one or more additives, such as antibacterial and/or antiviral compositions, such as silver, zinc, copper, organosilicone, tributyl tin, and organic compounds that contain chlorine, bromine, or fluorine compounds. The first or the second fibers may include waxes. Examples of wax include, but are not limited to, polyolefins, polyethylenes, functionalized waxes, such as amines, amides, fluorinated waxes, mixed fluorinated and amide waxes, such as esters, quaternary amines, carboxylic acids or acrylic polymer emulsion, chlorinated polyethylenes, natural or synthetic ester waxes, carnauba wax, paraffin, or the like, or any combination thereof. Such waxes may optionally be fractionated or distilled to provide specific cuts that meet certain viscosity and/or temperature criteria.

In certain embodiments, the first and second fibers discussed herein may be included as part of a filter device that traps or absorbs contaminants, such as a liquid filter, a gas filter for home and commercial air filtration, a medical grade filter such as a surgical mask, or other face covering, or the like. The filter device may be a mechanical filter, absorption filter, sequestration filter, ion exchange filter, reverse osmosis filter, surface filter, depth filter, or the like, and may be designed to remove many different types of contaminants from the air, water, or others.

In one such embodiment, the first and second fibers may be incorporated into an air filter that removes particles and contaminants from the air, such as a pleated mechanical air filter, a UV light filter, a washable filter, a medium filter, a spun glass filter, pleated, or unpleated air filters, active carbon filters, pocket filters, V-bank compact filters, filter sheets, flat cell filters, filter cartridges, and the like. The first and second fibers may be include in a filter media for the air filter and may be supported by a support layer, a scrim layer, or may be included in other layers or materials.

Conventional home and commercial air filters, such as pleated filters, may be typically rated by the filter's ability to capture particles between about 0.3 and 10 microns. This rating, referred to as a Minimum Efficiency Reporting Value or MERV is developed by the American Society of Heating, Refrigerating and Air Conditioning Engineers (ASHRAE). The MERV ratings range from 1-16, with higher values indicating higher efficiencies at trapping specific types of particles. It is also common to compare efficiency values in depending on particle sizes within the air stream during testing. E3, E2, and E1 values refer to particulate efficiency at 3-10 microns, 1-3 microns, and 0.3 to 1 micron, respectively.

The predicted MERV rating of the filter media discussed herein will vary based on many factors, including the types and sizes of fibers used in the filter media, the width of the filter media, the number and size of pleats (if any), face velocity and the like. Likewise, the pressure drop across the filter media will also depend on many factors, including those mentioned above.

In certain embodiments, the fibers may be ultrasonically bonded to form a filter media for a gas filter, such as an HVAC filter. In these embodiments, the ultrasonically bonded fibers decrease the pressure drop across the filter, while substantially maintaining the efficiency of a filter media in capturing contaminants in the E1, E2, and E3 particle groups. Thus, the MERV rating of the filter media may be substantially maintained, while the pressure drop across the filters may be reduced.

In embodiments, the filter media may have a pressure drop of from about 0.1 to about 4 mmH₂O at 10.5 fpm face velocity, and a predicted MERV rating of at least about 11 to about 16.

The filter media may include one or more support layers ultrasonically bonded to the fiber web. The support layer(s) may be ultrasonically bonded to the fibers without needle punching, which increases the stiffness of the support layer and the overall filter media. This may allow, for example, a pleated filter media to be created without a metal wire mesh or other structure designed to increase the stiffness of the media.

The support layer(s) may include a substrate, sheet, layer, film, apertured film, mesh, or other media. In certain embodiments, the support layer(s) may include a nonwoven substrate. The nonwoven substrate may include a structure of individual fibers or threads that may be interlaid, interlocked, or bonded together. Nonwoven fabrics may include sheets or web structures bonded together by entangling fiber or filaments (and by perforating films) mechanically, thermally, or chemically. They may be substantially flat, porous sheets that may be made directly from separate fibers or from molten plastic or plastic film. Examples of suitable nonwoven materials include, but are not limited to, fibers, layers, or webs that may be meltblown, spunbond or spunlace, heat-bonded, bonded carded, air-laid, wet-laid, co-formed, needle punched, stitched, hydraulically entangled or the like. In some embodiments, the support layer may be made of PET, polyamide (PA), and PP. In some embodiments, the support layer may be made of two polymers like bicomponent fibers such as CoPET/PET and HDPE/PET.

In certain embodiments, the support layer(s) may include a knitted and/or woven material. The knitted material may include any knitting pattern suitable for the desired application. Suitable knitted materials for filter applications include weft-knit, warp knit, knitted mesh panels, compressed knitted mesh, or the like. Suitable nonwoven materials for filter applications may include textile filter media, such as monofilament fabrics, multifilament fabrics, nylon mesh, polyester mesh, polypropylene mesh, or the like. Woven textiles may be used in, for example, mesh filter press cloths, nonwoven filter pads and other die-cut pieces, centrifuge filter bags, liquid filter bags, dust collector bags, bed dryer bags, rotary drum filters, filter belts, leaf filters, roll media, or the like.

In certain embodiments, the filter media may include a single support layer bonded to an outer surface of the fiber web. As shown in FIG. 2, a filter media 200 may include a carded web 210 of at least two dissimilar fibers, such as PP and acrylic (e.g., polyacrylate or acrylate polymer). The media 200 may further include a support layer 220 ultrasonically bonded to one of the outer surfaces of web 210.

In one embodiment, the support layer 220 may include a woven or nonwoven material, such as a scrim. The scrim layer may have a stiffness suitable for a pleated filter. In certain embodiments, the scrim layer may have a density of at least about 15 gsm after bonding with the fiber web, or at least about 40 gsm, or about 44 gsm, or at least about 50 gsm, or about 50 gsm to about 110 gsm.

In other embodiments, the filter media may include first and second support layers. As shown in FIG. 3, a filter media 300 may include a carded web 310 of dissimilar fibers, a first support layer 320 bonded to one surface of web 310 and a second support layer 330 bonded to the opposite surface of web 310. The first and second support layers 320, 330 may include any of the above materials and may include the same material. In certain embodiments, the first and second support layers 320, 330 may each include a spunbond scrim layer. In certain embodiments, the first scrim's basis weight may be from about 10 gsm to about 25 gsm, while the second scrim's basis weight may be from about 50 gsm to about 150 gsm.

FIG. 4 illustrates another embodiment of a filter media 400 comprising a carded web 410 of dissimilar materials and first and second support layers 420, 430 bonded to opposite sides of web 410. In this embodiment, first and second support layers 420, 420 comprise different materials. In certain embodiments, the first and second support layers 420, 420 each comprise a scrim layer.

In another embodiment, the first support layer may be bonded to the fibers and the second support layer may be bonded to the first support layer. As shown in FIG. 5, a filter media 500 may include a carded web 510 of dissimilar materials, a first support layer 520 bonded to one surface of web 510 and a second support layer 530 bonded to first support layer 520.

The contemplated fibers can be manufactured by any method, including, without limitation, melt spinning, wet spinning, dry spinning, meltblown, spunbond or spunlace, heat-bonded, carded, air-laid, wet-laid, extrusion, co-formed, stitched, hydraulically entangled or the like. Such methods are described in U.S. Pat. Nos. 4,406,950, 6,338,814, 6,616,435, 6,861,142, 7,252,493, 7,300,272, 7,309,430, 7,422,071, 7,431,869, 7,504,348, 7,774,077 9,522,357, 9,993,761 and United States Patent Publication No. 2009/266,759, the completed disclosures of which are hereby incorporated herein by reference for all purposes.

In one embodiment, the fibers may be carded prior to the ultrasonic bonding step. The system may include one carding machine or two carding machines disposed of in series with each other. Short fiber lengths may be processed through fiber opening, blending, and consolidation into a continuous fibrous web. Once the fibrous web has been formed from carding, the secondary process of ultrasonic bonding may be used to give the fibrous web integrity and strength.

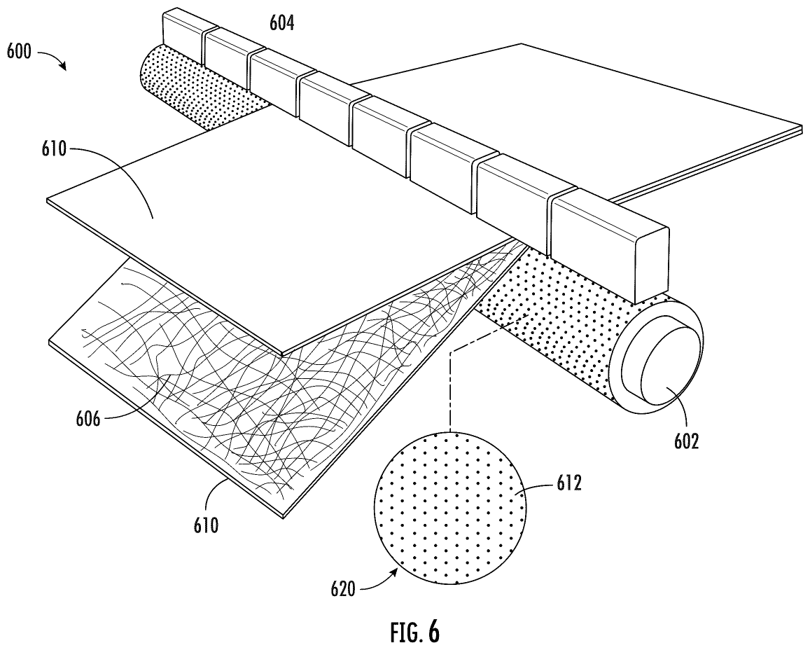

FIG. 6 illustrates an ultrasonic bonding device 600 that includes an anvil 602 and an ultrasonic horn 604. As shown, the first and second fibers 606 may be fed into the device 600 and high frequency ultrasonic vibrations may be applied to horn 604 to melt and/or bond the fibers 606 together. The inset in FIG. 6 illustrates the bonding attachment points 612 in the resulting fiber web 620, wherein the first and second dissimilar fibers may be bonded together by the ultrasonic energy. The attachment points 612 form indentations or dimples in the web 620 as the first and second fibers may be at least partially fused to each other at a plurality of discrete points across the web 620.

In addition, one or more support layers 610 may be bonded to the fiber web 620 in the same step (or in a separate step) as the bonding of the fibers 606. In one embodiment, a single support layer 610 may be ultrasonically bonded with the fibers 606 in the same step. In another embodiment, the single support layer 610 may be bonded to the fiber web 620 after the fibers have been bonded together. In yet another embodiment, multiple support layers 610 may be bonded to the fibers 606, or to the fiber web 620 in single or multiple steps. The bonding device 600 applies little to no pressure to the fibers 606 or the support layers 610. This allows the thickness or loft of the fibers 606 to be substantially maintained as the fibers 606 are bonded to each other. In addition, this allows the stiffness of the support layers 610 to be substantially maintained as they are bonded to the fiber web.

In certain embodiments, a self-supporting filter media is provided that may include first and second fibers ultrasonically bonded with each other to form a fiber web, and a second stiffer layer, such as a scrim, ultrasonically bonded to the fiber web. The scrim layer substantially maintains its original stiffness (prior to bonding) because it may not be needle-punched. Needling breaks the bond between fibers. This produces a self-supporting filter that may be “self-pleatable”, which means that the filter does not include a metal wire mesh or other supporting structure to form pleats. Ultrasonic bonding of the scrim may be done with or without needling of the fiber web. Alternatively, a stiff scrim may be ultrasonically bonded inline or offline.

In other embodiments, a medical grade filter, such as a facemask and CPAP (Continuous Positive Airway Pressure) media or the like, is provided. Medical grade filter media may typically include two scrim layers on both layers. The medical grade filter may include first and second fibers ultrasonically bonded with each other to form a fiber web. The filter may further include a first layer, such as a scrim, bonded to the first surface of the fiber web and a second stiffer material, such as a scrim, ultrasonically bonded to the second surface of the fiber web. The second scrim layer may substantially maintain its stiffness and may be bonded to the fibers in a single step to facilitate the manufacturing process.

In at least one embodiment, the filter may also include nanoparticles incorporated into the substrate or filter media. The nanoparticles may have at least one dimension less than 1 micron or less than 100 nm. The nanoparticles may increase the overall surface area within the filter media, which may increase its filtration efficiency and allows for the capture of submicron contaminants without significantly compromising other factors, such as pressure drop (i.e., air flow) through the filter. The nanoparticles may ensure that the efficiency of the filter remains relatively high even after the electrostatic charge decays over time. In addition, the bond between the fibers and the nanoparticles may be enhanced by the electrostatic charge, which may allow the nanoparticles to be dispersed in depth throughout the filter media.

In certain embodiments, the nanoparticles may be dispersed “in-depth” within the substrate. As used herein, the term “in-depth” means that the nanoparticles may be dispersed beyond a first surface of the substrate such that at least some of the nanoparticles may be disposed between the first and second opposing surfaces and into the internal structure of the substrate or media. In certain embodiments, the nanoparticles may be dispersed throughout substantially the entire media from the first surface to the opposing second surface. In other embodiments, the nanoparticles may be dispersed through a portion of the media from the first surface to a location between the first and second surfaces.

The nanoparticles may be chosen with different triboelectric properties relative to the first or second fibers in order to use the triboelectric effect to further enhance particle removal. With this method, the generated nanoparticles may be formed in an electrical field and may be less subject to contamination by chemicals that may moderate the triboelectric effect. Nanoparticles with different adsorption properties or surface charge characteristics than the first or second fibers may also be used (e.g., in oil or water filtration). This difference may be used to enhance or create localized electrical field gradients within the filter media to enhance particle removal. The nanoparticles and the fibers may have different wetting characteristics.

The nanoparticles may include any suitable material including, but not limited to, biosoluble glass, ceramic materials, acrylic, carbon, metal, alumina, polymers, such as nylon, polyethylene terephthalate (PET), polyvinyl chloride (PVC), polyolefin, polyacetal, polyester, cellulous ether, polyalkylene sulfide, poly(arylene oxide), polysulfone, modified polysulfone polymers, polyvinyl alcohol, polyamide, polystyrene, polyacrylonitrile, polyvinylidene chloride, polymethyl methacrylate, polyvinylidene fluoride, or the like, or any combination thereof.

In some embodiments, the nanoparticles may be bonded to the fibers via mechanical entanglement. This mechanical bond may be supplemented with an adhesive or binding agent. In certain embodiments, the nanoparticles may not be crimped (i.e., they do not include significant wavy, bent, curled, coiled sawtooth or similar shape associated with the nanoparticle in a relaxed state). In other embodiments, the nanoparticles may have a crimped body structure with a discrete length. For instance, when these crimped nanofibers having a discrete length are attached to the fiber, they entangle among themselves and with, onto, and around, the fiber with a firm attachment to form a modified fiber. In other embodiments, the attachment of the nanofibers to the micron fibers may be accomplished via electrostatic charge attraction and/or Van der Waals force attraction between the fibers and the nanoparticles. A more complete description of filter medias incorporating nanoparticles may be found in commonly assigned, co-pending International Patent Application No. PCT/US23/17921, filed Apr. 7, 2023, the complete disclosure of which is incorporated herein by reference in its entirely for all purposes.

The following numbered paragraphs disclose one or more exemplary variations of the subject matter of the application:

1. A filter media, comprising: a fibrous web comprising first electret fibers and second, dissimilar electret fibers configured for triboelectric charge; and wherein the first and second fibers are ultrasonically bonded together at an attachment point within the fibrous web.

2. The filter media of paragraph 1, wherein the fibrous web includes a plurality of attachment points, wherein the first and second fibers are bonded together at the plurality of attachment points.

3. The filter media of paragraph 2, wherein the plurality of attachment points forms indentations in or on an outer surface of the fibrous web.

4. The filter media of paragraph 2, wherein the first and second fibers are at least partially fused to each other at one or more of the plurality of attachment points.

5. The filter media of any one of paragraphs 1 to 4, wherein the first fibers comprise polypropylene (PP).

6. The filter media of any one of paragraphs 1 to 5, wherein the second fibers comprise acrylic.

7. The filter media of any one of paragraphs 1 to 6, wherein the fibrous web comprises: the second fibers in an amount of from about 40% to about 60% by weight of the filter media; and the first fibers in an amount of from about 40% to about 60% by weight of the filter media.

8. The filter media of any one of paragraphs 1 to 7, wherein the filter media has a loftiness of about 40 to about 300 mils.

9. The filter media of any one of paragraphs 1 to 8, wherein at least some of the first and second fibers comprise bicomponent fibers.

10. The filter media of any one of paragraphs 1 to 9, wherein the filter media comprises a pressure drop of about 0.1 to about 4 mmH₂O at 10.5 fpm face velocity, and a predicted MERV rating of from at least about 11 to about 16.

11. The filter media of any one of paragraphs 1 to 10, wherein the first and second fibers are carded fibers.

12. The filter media of any one of paragraphs 1 to 11, further comprising a support layer ultrasonically bonded to the fibrous web.

13. The filter media of paragraph 12, wherein the support layer has a density of at least about 40 gsm.

14. The filter media of paragraph 12 or 13, wherein the density of the support layer is between about 50 gsm to about 110 gsm.

15. An air filter product comprising the filter media of any one of paragraphs 1 to 14.

16. A self-supporting filter comprising the filter media of any one of paragraphs 1 to 14.

17. A self-pleating filter comprising the filter media of any one of paragraphs 1 to 14.

18. A medical grade filter, comprising: the filter media of any one of paragraphs 1 to 14; and a second support layer bonded to the fibrous web.

19. A face mask comprising the medical grade filter of paragraph 18.

20. A medical grade filter for use with a continuous positive airway pressure (CPAP) machine comprising the medical grade filter of paragraph 18.

21. A filter, comprising: a fibrous web comprising first electret fibers and second, dissimilar electret fibers configured for triboelectric charge, the first and second fibers being ultrasonically bonded together at an attachment point within the fibrous web; and a support layer ultrasonically bonded to the web.

22. The filter of paragraph 21, wherein the first and second fibers are triboelectrically charged with each other.

23. The filter of paragraph 21 or 22, wherein the support layer has a density of at least about 40 gsm.

24. The filter of any one of paragraphs 21 to 23, wherein the density of the support layer is between about 50 gsm to about 110 gsm.

25. The filter any one of paragraphs 21 to 24, wherein the support layer comprises a nonwoven material.

26. The filter of paragraph 25, wherein the nonwoven material comprises a scrim.

27. The filter of any one of paragraphs 21 to 23, wherein the support layer comprises spunbond polypropylene (PP).

28. The filter of any one of paragraphs 21 to 27, wherein the first fibers comprise polypropylene (PP).

29. The filter of any one of paragraphs 21 to 28, wherein the second fibers comprise acrylic.

30. The filter of any one of paragraphs 21 to 29, wherein the filter is self-pleatable.

31. The filter of any one of paragraphs 21 to 30, wherein the filter is self-supporting.

32. The filter of any one of paragraphs 21 to 31, wherein the filter is for a heating, ventilation, or air conditioning (HVAC) filter.

33. The filter of any one of paragraphs 21 to 32, further comprising a second support layer ultrasonically bonded to either of the fibrous web or the first support layer.

34. The filter of paragraph 33, wherein the first support layer is bonded to a first outer surface of the fibrous web, and the second support layer is bonded to a second outer surface of the fibrous web opposite the first outer surface.

35. The filter of paragraph 33, wherein the first support layer is bonded to an outer surface of the fibrous web, and the second support layer is bonded to the first support layer.

36. A medical grade filter comprising the filter of any one of paragraphs 21 to 35.

37. A face mask comprising the medical grade filter of paragraph 36.

38. A medical grade filter for use with a continuous positive airway pressure (CPAP) machine, comprising the medical grade filter of paragraph 36.

39. A method for manufacturing a filter media, the method comprising: providing first electret fibers; providing second, dissimilar electret fibers; and ultrasonically bonding the first and second fibers together to form a fibrous web.

40. The method of paragraph 39, further comprising carding the first and second fibers.

41. The method of paragraph 39 or 40, further comprising ultrasonically bonding a first support layer to the fibrous web.

42. The method of paragraph 41, wherein the first and second fibers and the first support layer are ultrasonically bonded to each other in a single step.

43. The method of paragraph 41 or 42, wherein the first support layer has a density of at least about 40 gsm after bonding with the web.

44. The method of any one of paragraphs 41 to 43, wherein the first support layer comprises a nonwoven material.

45. The method of paragraph 44, wherein the nonwoven material comprises a scrim.

46. The method of any one of paragraphs 41 to 45, wherein the first support layer comprises spunbond polypropylene (PP).

47. The method of any one of paragraphs 39 to 46, further comprising ultrasonically bonding the first and second fibers to each other without needle punching the first and second fibers.

48. The method of any one of paragraphs 41 to 47, further comprising ultrasonically bonding a second support layer to either the fibrous web or the first support layer.

49. The method of paragraph 48, further comprising ultrasonically bonding the first support layer to a first outer surface of the fibrous web, and ultrasonically bonding the second support layer to a second outer surface of the fibrous web opposite the first outer surface.

50. The method of paragraph 48, further comprising ultrasonically bonding the first support layer to a first outer surface of the fibrous web, and ultrasonically bonding the second support layer to the first support layer.

51. An air filter product formed from the method of any one of paragraphs 39 to 50.

52. A medical grade filter formed from the method of any one of paragraphs 39 to 51.

### EXAMPLES

The applicant conducted several tests of self-supporting triboelectric filter media. The filter media included a carded web of polypropylene (PP) fibers blended with acrylic fibers (50%/50% by weight of the carded web). Spunbond scrim layers were ultrasonically bonded to the carded web without needle punching. One of the layers included a scrim layer of PP having a density of about 15 gsm and the other layer included a stiffer scrim layer of PP with a density of about 44 gsm.

### Example 1

In the first example, a PP spunbond scrim having a density of about 15 grams per square meter (gsm) (labeled S) was ultrasonically bonded to one surface of the carded web (labeled A) and a PP spunbond scrim having a density of about 44 gsm (labeled SS) was ultrasonically bonded to the other surface of the carded web: (SS)-(A)-(S). The basis weight was measured in grams per square foot (gsf), grams per square meter (gsm), and ounces per square yard (osy). The results of this process are shown below in TABLE 1.

TABLE 1

Measured at 32 LPM

| | | | | Avg. | Avg. |
|---|---|---|---|---|---|
| | BASIS WEIGHT | | | Penetration | Resistance |
| | GSF | GSM | OSY | (%) | (mmH₂0) |
| SS-A-S | 13.4 | 144.3 | 4.3 | 5.8 | 0.7 |

As the PP spunbond scrim (SS) was a pleat support, the penetration demonstrated that the product is feasible for filtration applications at a reasonable resistance with self-pleating ability. It is preferred for medical applications, such as CPAP filters, to have both layers covered with a scrim.

### Example 2

In a second example, a PP spunbond scrim having a density of about 15 gsm (labeled S) was ultrasonically bonded to one surface of the carded web (labeled A) and a PP spunbond scrim having a density of about 44 gsm (labeled SS) was ultrasonically bonded to the scrim(S) layer: (SS)-(S)-(A). The results of this process are shown below in TABLE 2.

TABLE 2

Measured at 32 LPM

| | | | | Avg. | Avg. |
|---|---|---|---|---|---|
| | BASIS WEIGHT | | | Penetration | Resistance |
| | GSF | GSM | OSY | (%) | (mmH₂0) |
| SS-S-A | 13.5 | 145.7 | 4.3 | 4.2 | 0.7 |

Similar to Example 1, the penetration supports the application of the product for filtration applications. It should be appreciated that both PP spunbond scrims were or had self-pleating abilities.

While the devices, systems, and methods have been described in detail herein in accordance with certain preferred embodiments thereof, many modifications and changes therein may be effected by those skilled in the art. Accordingly, the foregoing description should not be construed to be limited thereby but should be construed to include such aforementioned obvious variations and be limited only by the spirit and scope of the following claims.

What is claimed is:

1. A filter media, comprising:
a fibrous web comprising first electret fibers and second, dissimilar electret fibers configured for triboelectric charge; and
wherein the first and second fibers are ultrasonically bonded together at a plurality of attachment points within the fibrous web.

2. The filter media of claim 1, wherein the first fibers comprise polypropylene (PP).

3. The filter media of claim 1, wherein the second fibers comprise acrylic.

4. The filter media of claim 1, wherein the fibrous web comprises:
the second fibers in an amount of from about 40% to about 60% by weight of the filter media; and
the first fibers in an amount of from about 40% to about 60% by weight of the filter media.

5. The filter media of claim 1, wherein the filter media comprises one or more of:
a loftiness of about 40 to about 300 mils;
a pressure drop of about 0.1 to about 4 mmH₂O at 10.5 fpm face velocity; and
a predicted MERV rating of from at least about 11 to about 16.

6. The filter media of claim 1, wherein at least some of the first and second fibers comprise bicomponent fibers.

7. The filter media of claim 1, wherein the first and second fibers are carded fibers.

8. The filter media of claim 1, further comprising a support layer ultrasonically bonded to the fibrous web.

9. The filter media of claim 8, wherein the support layer has a density of at least about 40 gsm.

10. An air filter product, a self-supporting filter, or a self-pleating filter, comprising the filter media of claim 1.

11. A medical grade filter, comprising:
the filter media of claim 8; and
a second support layer bonded to the fibrous web.

12. A face mask or a medical grade filter for use with a continuous positive airway pressure (CPAP) machine, comprising the medical grade filter of claim 11.

13. A filter, comprising:
the filter media of claim 1; and
a support layer ultrasonically bonded to the fibrous web of the filter media.

14. The filter of claim 13, wherein the support layer comprises a nonwoven material.

15. The filter of claim 13, wherein the support layer comprises spunbond polypropylene (PP).

16. The filter of claim 13, wherein the filter is self-pleatable or self-supporting.

17. The filter of claim 13, wherein the support layer is a first support layer bonded to a first outer surface of the fibrous web, and further comprising a second support layer, wherein the second support layer is ultrasonically bonded to a second outer surface of the fibrous web opposite the first outer surface or to the first support layer.

18. A medical grade filter, comprising the filter of claim 13.

19. A face mask or a medical grade filter for use with a continuous positive airway pressure (CPAP) machine, comprising the medical grade filter of claim 18.

20. A method for manufacturing a filter media, the method comprising:
providing first electret fibers;
providing second, dissimilar electret fibers; and
ultrasonically bonding the first and second fibers together to form a fibrous web.

21. The method of claim 20, further comprising ultrasonically bonding a first support layer to the fibrous web.

22. The method of claim 21, wherein the first and second fibers and the first support layer are ultrasonically bonded to each other in a single step.

23. The method of claim 21, wherein the first support layer comprises a nonwoven material, optionally, the first support layer comprise spunbond polypropylene (PP).

24. The method claim 20, wherein ultrasonically bonding the first and second fibers together does not include needle punching the first and second fibers.

25. The method of claim 21, further comprising ultrasonically bonding a second support layer to either the fibrous web or the first support layer.
